(19) United States
(12) Patent Application Publication
(10) Pub. No.: US 2016/0151578 A1
(43) Pub. Date: Jun. 2, 2016

(54) DRIVE ASSEMBLY FOR A DRUG DELIVERY DEVICE

(71) Applicant: SANOFI, Paris (FR)

(72) Inventors: Tom Oakley, Cambridge Cambridgeshire (GB); Stuart Milne, Buckden St. Neots, Cambridgeshire (GB)

(21) Appl. No.: 14/904,738

(22) PCT Filed: Jul. 17, 2014
(86) PCT No.: PCT/EP2014/065327

§ 371 Date: Jan. 13, 2016

(30) Foreign Application Priority Data
Jul. 17, 2013 (EP) 13176846.7

(51) Int. Cl. A61M 5/315

(52) U.S. Cl. CPC A61M 5/31551

(57) ABSTRACT

A drive assembly for a drug delivery device (100) is presented. The drive assembly comprises a housing (4) having a proximal end (18) and a distal end (19) and a main drive mechanism comprising a drive member (5), a piston rod (7) and a resilient member (13). The main drive mechanism being configured such that, in a setting mode of operation, the resilient member (13) is biased when a dose of a drug (35) is set and in a dispensing mode of operation, the set dose is dispensed, wherein the drive member (5) drives the piston rod (7). The drive assembly further comprises an auxiliary drive mechanism further comprising an auxiliary drive member (26) which is coupled to the drive member (5). In the dispensing mode of operation, the auxiliary drive mechanism can be actuated by a user via a movement of the auxiliary drive member (26) with respect to the housing (4), thereby mechanically assisting the dispensing action of the main drive mechanism.

The present disclosure relates to a drive assembly for a drug delivery device, e.g. an injector-type device such as a pen-type device, and to the drug delivery device.

Drug delivery devices are, for example, known from EP 1795219 B1 and EP 1351732 B1.

It is an object of the present disclosure to provide an improved drive assembly for a drug delivery device which preferably facilitates operability of the drug delivery device.

This object is achieved by the subject-matter of the independent claim. Advantageous embodiments and refinements are subject-matter of the dependent claims.

One aspect of the present disclosure relates to a drive assembly for a drug delivery device comprising a housing having a proximal end and a distal end. The drive assembly further comprises a main drive mechanism comprising a drive member, a piston rod being coupled to the drive member and a resilient member. The main drive mechanism is configured such that, in a setting mode of operation, the resilient member is biased when a dose of drug is set and in a dispensing mode of operation the set dose is dispensed from the drug delivery device in a dispensing action, wherein the drive member drives the piston rod with respect to the housing. The dispensing action is at least partially driven by the resilient member. The drive assembly further comprises an auxiliary drive mechanism comprising an auxiliary drive member which is coupled to the drive member. The auxiliary drive mechanism is configured such that, in the dispensing mode of operation, the auxiliary drive mechanism can be actuated by a user via a movement of the auxiliary drive member with respect to the housing, thereby mechanically assisting the dispensing action of the main drive mechanism. A further aspect of the present disclosure relates to a drug delivery device comprising the drive assembly.

The “distal end” of the drug delivery device or a component of the drug delivery device shall mean the end which is closest to the dispensing end of the drug delivery device.

The “proximal end” of the drug delivery device or a component of the drug delivery device shall mean the end which is furthest away from the dispensing end of the drug delivery device.

The drug delivery device comprises a longitudinal axis. Preferably, the longitudinal axis extends through the proximal end and the distal end. The proximal and the distal end may be spaced apart from each other along the longitudinal axis.

The dose size of the drug delivery device may be user-settable.

Preferably, the main drive mechanism is configured to dispense a set dose from the drug delivery device in the dispensing action even without a contribution of the auxiliary drive mechanism. The auxiliary drive mechanism, however, provides the advantage that e.g. the injection speed may be varied and/or controlled by the user during the dispensing action. By manipulating, e.g. pressing an actuation member such as a button, the user of the drive assembly is enabled to transfer force via the auxiliary drive mechanism to the piston rod to directly assist or support the main drive mechanism in dispensing a dose of drug. Moreover, the user is given a haptic feedback that a dose is currently being dispensed from the drug delivery device. This is because the drive assembly may be configured such that the user may e.g. directly feel the advance of the drive mechanism during a dispensing action, whereby the intensity by which the user manipulates or presses the actuation member determines the intensity by which the user directly assists or supports the main drive mechanism in dispensing a dose of drug.

In an embodiment, the drive member is coupled to the resilient member. This provides the advantage that a movement of the resilient member may be transferred to the drive member and/or the resilient member may drive the drive member.

In an embodiment, in the dispensing mode of operation, the drive member is rotatable in a first direction with respect to the housing. According to this embodiment, a rotation of the drive member may be transferred to the piston rod.

In an embodiment the drive assembly comprises an actuation member which is coupled to the auxiliary drive member. The actuation member is axially moveable with respect to the housing. The actuation member may be directly manually activated by the user in order to trigger the dispensing action of the drug delivery device. Thereby, preferably, the actuation member is moved or depressed distally with respect to the housing. According to this embodiment, the auxiliary drive member may be moved distally via the actuation member. Preferably, there is only one actuation member for triggering a dispensing action or an injection and for the triggering or actuation of the auxiliary drive mechanism.

In an embodiment, the drive assembly is configured such that when, in or starting from the setting mode of operation, wherein the dose is set, the actuation member is moved distally with respect to the housing, the drive assembly is switched from the setting mode to the dispensing mode of operation.

In an embodiment, the drive assembly is configured such that when, in the setting mode of operation, the actuation member is moved distally with respect to the housing, the auxiliary drive member is moved distally with respect to the housing, thereby transferring a force to the drive member in order to drive the drive member in addition to the force provided by the resilient member. Via the auxiliary drive member, the user may advantageously assist or support the main drive mechanism when he moves the actuation member distally with respect to the housing, whereby also a haptic feedback to the user is provided which, e.g. gives a hint to the user when the dispensing action is completed. Furthermore, the user may accelerate the dispensing action when the auxiliary drive member is moved distally with respect to the housing, depending on how fast or hard the actuation member is depressed or moved distally.

In an embodiment, the drive assembly is configured such that when, in the setting mode of operation, a dose is set in a setting action, the auxiliary drive member is rotated with respect to the drive member in a second direction, opposite to the first direction.

Thereby, when a dose is set, it is advantageously ensured that the auxiliary drive member is brought into a relative position with respect to the drive member that—during a subsequent dispensing action—the auxiliary drive member may again be moved distally by the user in order to drive the drive member once again. Preferably, the drive member is axially locked with respect to the housing such that, when the auxiliary drive member is rotated with respect to the drive member, it is also moved proximally with respect to the drive member and with respect to the housing.

A rotation of components of the drive assembly in the first direction may relate to a rotation of said components during a dispensing action in the dispensing mode of operation.

A rotation of components of the drive assembly in the second direction may relate to a rotation of said components during a setting action in the setting mode of operation.

In an embodiment, when in or starting from the setting mode of operation, wherein the dose is set, the actuation member is moved distally with respect to the housing, the drive member rotates in the first direction with respect to the auxiliary drive member. During said movement in the first direction, the user is advantageously given the opportunity to transfer the force to the drive member in order to drive the drive member in addition to the force provided by the resilient member.

In an embodiment, distal movement of the auxiliary drive member can be transferred into a rotation of the drive member, e.g. in the first direction with respect to the housing.

In an embodiment, the drive member is threadedly engaged to the auxiliary drive member. Thereby, it is expediently achieved that a distal movement of the auxiliary drive member may be transferred into a rotation of the drive member in the first direction with respect to the housing. Said threaded engagement is expediently not self-locking.

In an embodiment, the drive member is rotationally locked with respect to the piston rod. The piston rod is threadedly engaged to a housing component of the drive assembly. The housing component may be a nut member. According to this embodiment, it is achieved that a rotation of the drive member in the first direction may be converted into a rotation of the piston rod with respect to the housing component which rotation, in turn, is converted into an axial displacement of the piston rod, preferably a distal movement of the piston rod, whereby a dose of drug may be dispensed from the drug delivery device.

In an embodiment, the drug delivery device comprises a cartridge retaining a plunger, wherein the drug delivery device is configured such that during the dispensing action the piston rod is moved distally with respect to the cartridge, thereby advancing the plunger distally, whereby a dose of drug is dispensed from the drug delivery device.

In an embodiment, the drug delivery device comprises a needle or a needle assembly. Through said needle or needle assembly, a drug or medical substance which may be retained in the cartridge, can be dispensed from the drug delivery device.

In an embodiment, the drive assembly is configured such that when, in or starting from the setting mode of operation, wherein the dose is set, the actuation member is depressed by the user, the auxiliary drive member mechanically assists the rotation of the drive member. The actuation member may be depressed by the user in order to be moved distally with respect to the housing. Thereby, the piston rod is moved distally with respect to the housing and a dose of drug may be dispensed from the drug delivery device. If the user only slightly depresses the actuation member, the main drive mechanism or the mentioned rotation of the drive member may possibly be not or not significantly assisted. In this case, the dispensing action may substantially be driven by the main drive mechanism. The assist or said rotation of the drive member, preferably relates to a dispensing action, wherein the user depresses the actuation member such that he notices or feels the mentioned haptic feedback of the main drive mechanism.

In an embodiment, the drive assembly comprises a dose member. The dose member may be rotationally locked with respect to the drive member, but preferably not rotated. Thereby, it is achieved that a rotation of the dose member is transferred to or drives a rotation of the drive member with respect to the housing.

In an embodiment, the drive assembly is configured such that, during a setting action, the dose member is moved proximally with respect to the drive member.

In an embodiment, the dose member comprises a last dose feature which is arranged to engage a piston rod feature of the piston rod when, in the setting mode of operation, the axial distance by which the dose member is moved proximally is less than the axial distance between the last dose feature and the piston rod feature. With this embodiment, it may be achieved that a user is prevented from setting a dose of drug which is greater than the available volume of drug left in the cartridge.

In an embodiment, the axial distance between the piston rod feature and the last dose feature is indicative for an amount of drug remaining to be dispensed from the drug delivery device, wherein, when the last dose feature and the piston rod feature are engaged, the dose member is prevented from being moved further proximally. With this embodiment, it is achieved that a user may be prevented from setting a dose of drug greater than the available volume in the cartridge. In other words, a user is thereby prevented from setting a dose which is greater than that one which is actually dispensable from the drug delivery device. User safety is increased in this way.

In an embodiment, the drive assembly comprises a ratchet member. The ratchet member is coupled to the housing, preferably via a releasable uni-directional coupling. According to this embodiment, a biasing of the resilient member is enabled, whereby energy provided by the biased resilient member may be stored. In other words, it may be achieved that during a setting action, the resilient member does not instantaneously relax into its initial state. Said releasable uni-directional coupling preferably only effects in one direction such that, when the coupling is established, the ratchet member can only be moved and/or rotated in one direction with respect to the housing.

In an embodiment, the ratchet member is threadedly engaged to the housing.

In an embodiment, the housing further comprises a setting stop and/or a dispensing stop. Under certain conditions, these stops preferably prevent a rotation of the ratchet member with respect to the housing in the proximal and in the distal direction, respectively. Said stops may engage with corresponding stops on the ratchet member, respectively. A corresponding setting stop may, e.g. be arranged at a proximal end of an outer thread of the ratchet member and a corresponding dispensing stop may be arranged at a distal end of said thread. Thus, between certain limits, the ratchet member may be rotationally moveable with respect to the housing.

In an embodiment, the drive assembly is configured such that, in the setting mode of operation, the releasable uni-directional coupling is established and, during a setting action, the ratchet member is rotated in the second direction with respect to the housing. When the ratchet member is rotated in the second direction, it preferably moves proximally with respect to the housing and with respect to the drive member. By means of a rotation of the ratchet member in the second direction with respect to the housing, the resilient member is preferably biased.

In an embodiment, in the dispensing mode of operation, the releasable uni-directional coupling is disengaged and the ratchet member rotates in the first direction with respect to the housing. Thereby, the ratchet member may expediently drive further components such as the dose member and the drive member during a dispensing action. The ratchet member preferably transfers the rotation in the first direction to the dose member in the first direction with respect to the housing such that the drive member is also rotated in the first direction with respect to the housing, as the dose member and the drive member are, preferably rotationally locked with respect to each other.

In an embodiment, the drive assembly comprises a drive coupling which is configured such that, in the setting mode of operation, the ratchet member is free to rotate with respect to the dose member and, in the dispensing mode of operation, the ratchet member is rotationally locked with respect to the dose member such that, during a dispensing action, the dose member is rotated in the first direction with respect to the housing. The drive coupling may provide a sort of clutch mechanism. When the drive coupling is released, the ratchet member is free to rotate with respect to the dose member. When the drive coupling is engaged, the ratchet member and the dose member are rotationally locked with respect to each other such that a rotation of the ratchet member is transferred to the dose member. Particularly, this embodiment allows that energy provided by the biased resilient member is stored by the releasable uni-directional coupling, whereby said energy may selectively be used to drive the dispensing of a dose of drug from the drug delivery device. Particularly, a rotation of the ratchet member in the first direction may drive a rotation of the dose member, the drive member and the piston rod in the first direction such that the piston rod advances distally due to its engagement to the housing component. Thereby, a dose of drug may be dispensed from the drug delivery device. In the dispensing mode of operation, a force provided by the biased resilient member may be exerted to the ratchet member in order to dispense a dose of drug, as described. Said force may relate to that one exerted or provided by the main drive mechanism and/or the auxiliary drive mechanism.

In an embodiment, the drive assembly comprises a clutch element and a clutch spring. The clutch spring is retained between the ratchet member and the clutch element. The clutch spring tends to move the ratchet member and the clutch element away from each other. By means of the clutch element and the clutch spring, functionality may be provided by which a spline engagement of the ratchet member and the dose member is engaged when the dose member is moved distally with respect to the ratchet member.

The clutch element may be rotatable with respect to the dose member.

In an embodiment, the ratchet member comprises ratchet splines and the dose member comprises dose member splines, whereby the ratchet splines and the dose member splines form the drive coupling.

In an embodiment, the drive assembly is configured such that when, in or starting from the setting mode of operation, the actuation member is moved distally with respect to the housing, the clutch element is moved distally such that the drive coupling is engaged. The drive assembly is preferably configured such that, when the clutch element is moved distally, the dose member is moved distally with respect to the ratchet member. Preferably, thereby, the ratchet splines are moved into engagement of the dose member splines.

In an embodiment, the resilient member is a torsion spring, e.g. a torsion coil spring, wherein the resilient member is connected to the ratchet member such that it is biased when the ratchet member is rotated in the second direction with respect to the housing. In this way, it is embodied most expediently that a rotation of the ratchet member in the second direction during a setting action is, during a dispensing action, transferred into a rotation of the piston rod in the first direction in order to dispense a dose of drug from the drug delivery device.

In an embodiment, the drive assembly comprises a dose setting member, wherein the actuation member is axially movable with respect to the dose setting member. By means of the actuation member, the user may preferably trigger the switching from the setting mode to the dispensing mode of operation as well as e.g. the release or the disengagement of the releasable uni-directional coupling.

In an embodiment, the drive assembly is configured such that, in the setting mode of operation, the dose setting member is rotationally locked with respect to the ratchet member and when, in or starting from the setting mode of operation, the actuation member is moved distally with respect to the housing and the dose setting member, the rotational locking between the ratchet member and the dose setting member is released and the dose setting member is rotationally locked with respect to the housing by an actuation member feature of the actuation member.

In an embodiment, the drive assembly is configured such that during a dose setting action, the dose setting member is rotated by the user in the second direction with respect to the housing in order to set the dose. By means of the dose setting member, the user may advantageously directly manually set a dose by gripping and rotating the dose setting member with respect to the housing.

In an embodiment, the drive assembly is configured such that, in the setting mode of operation, the releasable uni-directional coupling is releasable when the dose setting member is rotated in the second direction with respect to the housing. Preferably, the user may release or overcome the friction of the releasable uni-directional coupling in that he rotates the dose setting member in the second direction with respect to the housing.

In an embodiment, the piston rod comprises a metal and a rectangular cross-section. The use of sheet metal provides the advantage that the mass of material used for the piston rod can be reduced, thus saving material costs. Moreover, the processes involved in the manufacture of sheet metal are typically less time-consuming and, therefore, cheaper. Additionally, less waste of material is created, as compared to conventional machining processes.

Features which are described herein above and below in conjunction with different aspects or embodiments may also apply for other aspects and embodiments.

Further features and advantages of the subject matter of this disclosure will become apparent from the following description of the exemplary embodiment in conjunction with the figures, in which:

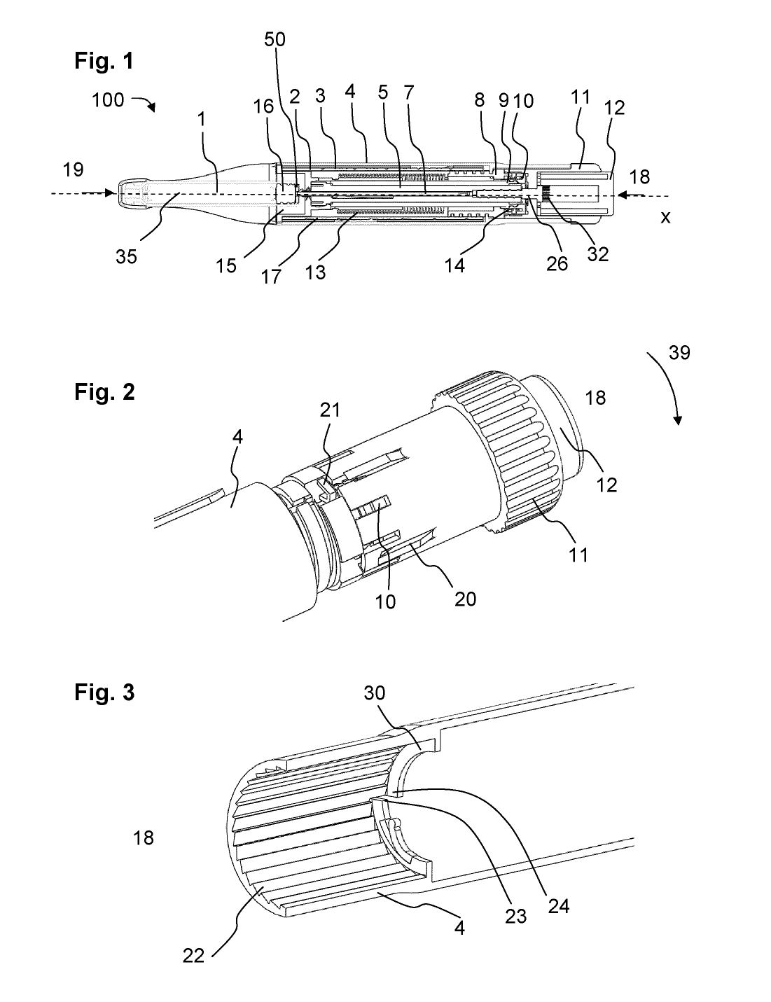

FIG. 1 shows a sectional side view of a drug delivery device comprising a drive mechanism.

FIG. 2 shows a perspective view of parts of a proximal end of the drug delivery device.

FIG. 3 shows a sectional perspective view of a proximal end of a housing of the drug delivery device.

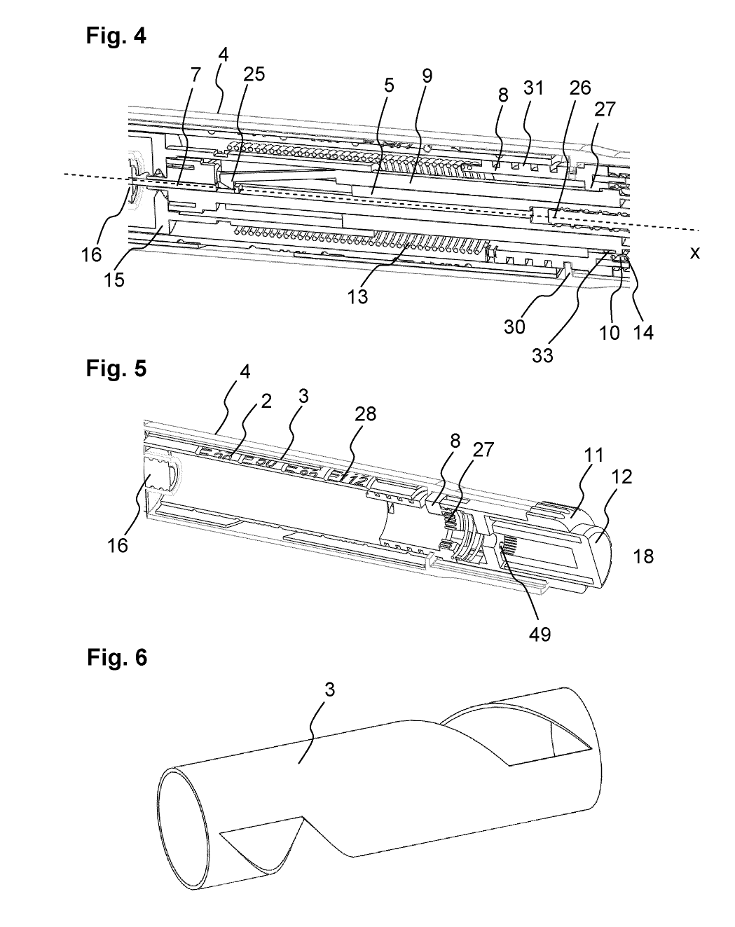

FIG. 4 shows a sectional view of inner components of the drug delivery device.

FIG. 5 shows a sectional perspective view of inner components of the drug delivery device.

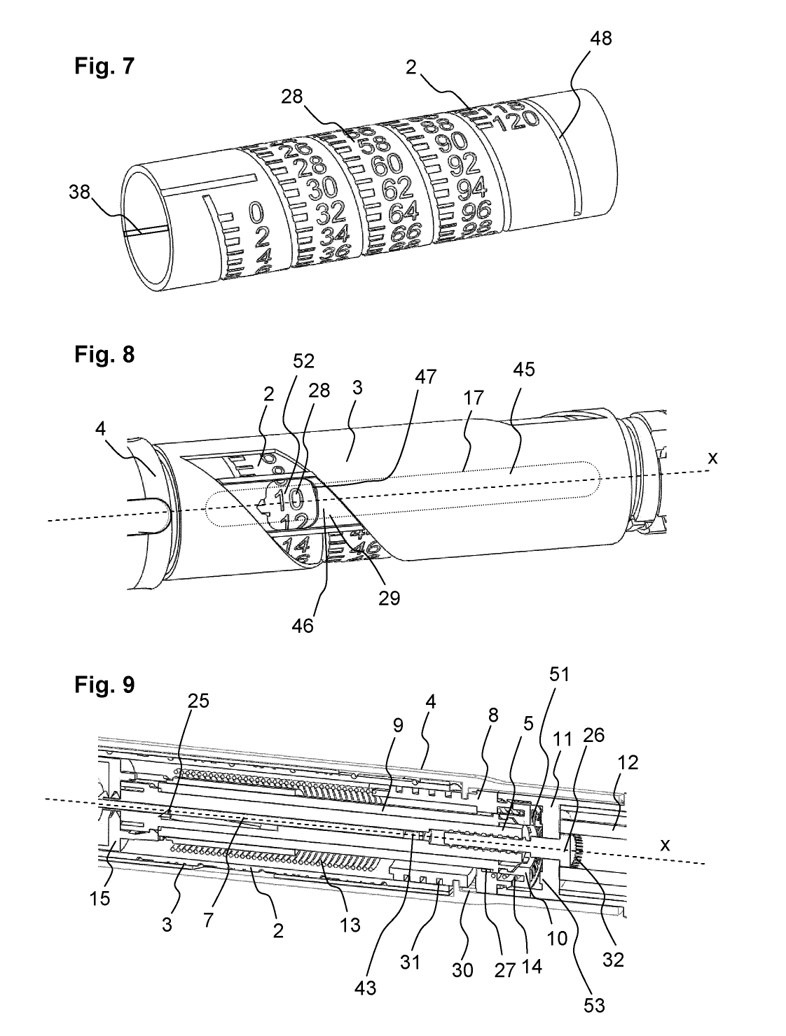

FIG. 6 shows a perspective view of a selection member of the drug delivery device. FIG. 7 shows a perspective view of an indication member of the drug delivery device.

FIG. 8 shows a perspective view of a display assembly of the drug delivery device.

FIG. 9 shows a sectional view of inner components of the drug delivery device.

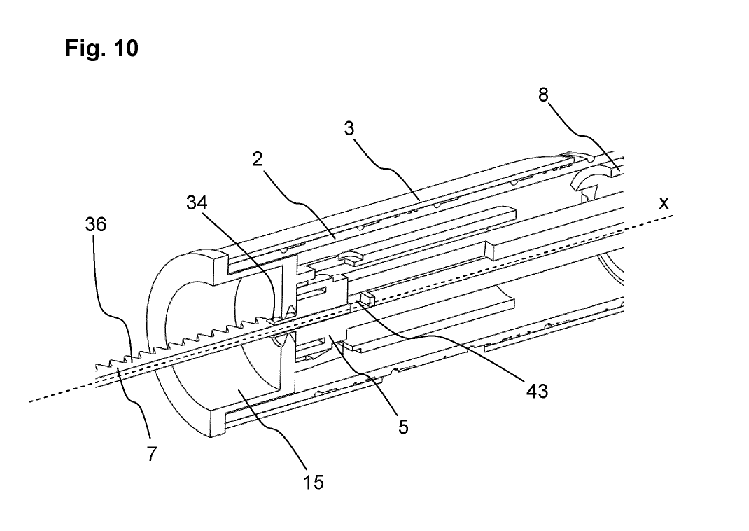

FIG. 10 shows a sectional perspective view of inner components of the drug delivery device.

Like elements, elements of the same kind and identically acting elements may be provided with the same reference numerals in the figures. Additionally, the figures may not be true to scale. Rather, certain features may be depicted in an exaggerated fashion for better illustration of important principles.

FIG. 1 shows a drug delivery device 100 comprising a drive assembly. A plurality of components and functionalities which are described herein relate to said drive assembly. The drive assembly, in turn, comprises a main drive mechanism and an auxiliary drive mechanism which are described further below.

The drug delivery device 100 comprises a proximal end 18 and a distal end 19. The drug delivery device 100 further comprises a main longitudinal axis x which is disposed between the proximal end 18 and the distal end 19. The drug delivery device 100 further comprises a housing 4. The housing 4 may house or retain further components of the drug delivery device 100. The housing 4 may be a body. The drug delivery device 100 further comprises a window 17. The window 17 may comprise an elongate shape with a longitudinal axis being aligned with the longitudinal axis x. The drug delivery device 100 further comprises an indication member 2 which provides pieces of information to be displayed through the window 17 of the drug delivery device 100. The indication member 2 may be an indication sleeve. Said information to be displayed may pertain to the size of a dose of a drug 35 to be dispensed from the drug delivery device 100. Therefore, the indication member 2 may comprise indicia (cf. 28 in FIG. 7) which indicate units or quantities of drug 35. The drug delivery device 100 further comprises a selection member 3 which may be a selection sleeve. The selection member 3 may mask information provided by the indication member 2 to be displayed through the window 17. The indication member 2 and the selection member 3, preferably, comprise an elongate shape. Preferably, the indication member 2 and the selection member 3 are rotatable around the longitudinal axis x with respect to the housing 4. A longitudinal axis of the indication member 2 and a longitudinal axis of the selection member 3 are preferably aligned with the longitudinal axis x. Said components are further preferably not axially moveable with respect to the housing.

The drug delivery device 100 further comprises a cartridge 1 containing the drug 35. The cartridge may be a 1.5 ml cartridge or a 3.0 ml cartridge. The cartridge may also contain different volumes of drug. A plunger 16 is retained at the proximal end of the cartridge 1. In the depicted situation, drug 35 has not yet been dispensed from the drug delivery device 100. The drug delivery device 100 further comprises a drive member 5 which is provisioned to drive the piston rod 7 during a dispensing action, i.e. when a set dose of drug 35 is dispensed from the drug delivery device 100. The drive member 5 comprises an elongate shape with a longitudinal axis being aligned with the longitudinal axis x. The drive member 5 is preferably rotatable in a first direction with respect to the housing 4.

The drug delivery device 100 further comprises a piston rod 7. The piston rod 7 comprises a distal termination 50 which may be rotatable with respect to the piston rod 7 and which is preferably arranged axially next to the plunger 16. The drive member 5 is rotationally locked with respect to the piston rod 7. Both components comprise an elongate shape with a longitudinal axis preferably aligned to the longitudinal axis x. Preferably, the drive member 5 is rotatable but axially constrained with respect to the housing 4. The piston rod 7 is threadedly engaged to a nut member 15 which is fixed to the housing 4 or unitarily formed by the housing 4. To this effect, the piston rod comprises an outer thread 36 (cf. FIG. 10) matching with an inner thread (not explicitly indicated) of the nut member 15. During dispensing of a set dose of drug 35, the drive member 5 preferably interacts with the piston rod 7 such that the piston rod 7 is moved distally due to the threaded engagement with the nut member 15. Consequently, the plunger 16 is advanced with respect to the cartridge 1 in the distal direction. Thereby, drug 35 is preferably dispensed from the drug delivery device 100.

The drug delivery device 100 may further comprise a needle or a needle assembly (not explicitly indicated). Said needle or needle assembly is preferably fluidly connected to the cartridge 1 such that during a dispensing action, drug 35 may be dispensed through the needle or the needle assembly.

The drug delivery device 100 may further comprise a cap (not shown) covering e.g. the distal end 19 and/or a needle hub to which the needle may be mounted. The cap may be provisioned to protect the distal end 19 of the drug delivery device 100 from contamination.

The drug delivery device 100 further comprises a ratchet member 8 comprising a sleeve-like shape. The ratchet member 8 comprises an outer thread 31 by which it is threadedly engaged to the housing 4. The drug delivery device 100 further comprises a resilient member 13. The resilient member 13 is a torsion spring. The resilient member is preferably connected to the housing 4 and to a distal end of the ratchet member 8. By a rotation (cf. second direction below and 39 in FIG. 2) of the ratchet member 8 with respect to the housing 4, the resilient member 13 is biased. The ratchet member 8 is furthermore coupled to the housing 4 via a releasable uni-directional coupling.

Due to its threaded engagement with the housing 4, the ratchet member 8 ratchets the resilient member 13 such that spring energy of the resilient member 13 is stored during a setting action, i.e. when, the setting mode of operation, a dose of drug 35 is set. The ratchet member 8 may prevent a relaxation of the resilient member 13 when a dose is set. The resilient member 13 preferably relates to the main drive mechanism of the drive assembly. Particularly, the resilient member 13 may drive the main drive mechanism.

The ratchet member 8 may be a display driver which drives the indication member 2 during an operation (e.g. a setting or dispensing action) of the drive assembly. To this effect, the indication member 2 may rotationally locked with respect to the ratchet member 8 via an axial rib 38.

The drug delivery device 100 further comprises a dose member 9 which is rotationally locked with respect to the drive member 5. The dose member 9 comprises an elongate shape with a longitudinal axis aligned with the longitudinal axis x. Preferably, the dose member 9 is axially moveable with respect to the housing 4. The dose member 9 is provisioned to transfer a torque or rotation to the drive member 5 during an operation of the drug delivery device 100. Particularly, in a dispensing mode of operation, the dose member 9 rotates along with the drive member 5 in a first direction with respect to the housing 4.

The drug delivery device 100 further comprises an actuation member 12 which is arranged at a proximal end 18 of the drug delivery device 100. The actuation member 12 is axially movable with respect to the housing 4. Particularly, a user can depress the actuation member 12 with respect to the housing 4. The actuation member 12 may be a button. When the actuation member 12 is moved distally with respect to the housing such as depressed by the user, a drive assembly of the drug delivery device 100 is preferably switched from the setting mode to the dispensing mode of operation.

The drug delivery device 100 further comprises a dose setting member 11 by which the user of the drug delivery device 100 may set a dose of drug 35 which is to be dispensed from the drug delivery device 100. To this effect, the user may manually rotate the dose setting member 11 in the second direction being opposite to the first direction with respect to the housing 4. In the setting mode of operation, the dose setting member 11 is rotationally locked with respect to the ratchet member 8.

The actuation member 12 is partially retained within the dose setting member 11. The actuation member 12 is rotationally locked but axially moveable with respect to the dose setting member 11.

The drug delivery device 100 further comprises an auxiliary drive member 26 forming part of the auxiliary drive mechanism. The auxiliary drive member has an elongate shape with a longitudinal axis being aligned with the longitudinal axis x. The drive assembly is configured such that when, in the setting mode of operation, a dose is set, the auxiliary drive member 26 is rotated in the second direction with respect to the drive member 5. In the dispensing mode of operation, the auxiliary drive member preferably mechanically assist the rotation of the drive member 5, whereby the piston rod 7 is moved distally with respect to the housing 4 due to the mentioned thread engagement with the nut member 15. To this effect, the drive member 5 is threadedly engaged to the auxiliary drive member 26.

When the actuation member 12 is depressed, the rotational coupling between the dose setting member 11 and the ratchet member 8 is released.

The actuation member 12 may have at least one actuation pin 49 which interacts with the clutch element 10 to move it axially relative to the dose setting member 11, therebys disengaging the proximal teeth 51 from the distal teeth 53.

The drug delivery device 100 further comprises a clutch element 10 and a clutch spring 14. The clutch spring 14 is retained between the ratchet member 8 and the clutch element 10, thereby tending to move the ratchet member 8 and the clutch element 10 away from each other. When in the setting mode of operation, the actuation member 12 is moved proximally with respect to the housing 4, and the clutch element 10 is moved distally with respect to the housing 4 thereby disengaging the releasable unidirectional coupling.

FIG. 2 shows parts of the proximal end 18 of the drug delivery device 100 without the housing 4. It is shown that the ratchet member 8 comprises ratchet features 21 (only one shown in FIG. 2) which—when assembled in the drug delivery device 100 (cf. FIG. 1)—engages teeth 22 of the housing 4 (cf. FIG. 3), thereby forming the releasable uni-directional coupling between the ratchet member 8 and the housing 4. Furthermore, the actuation member 12 comprises actuation features 20. The drive assembly is configured such that when, in the setting mode of operation, the actuation member 12 is moved distally with respect to the housing 4, the actuation features 20 are pushed radially outwards through slots (not explicitly indicated) of dose setting member 11. Consequently, the actuation features 20 engage with the teeth 22 on the inside of the housing 4 (cf. FIG. 3). Thereby, the actuation member 12 rotationally locks the dose setting member with respect to the housing 4.

During a setting action, the ratchet feature 21 may slide over teeth of the teeth 22. Thereby, the ratchet feature 21 may be slightly deflected inwards until it has passed the respective tooth. Preferably, the teeth 22 is configured such that—provided the releasable uni-directional coupling is engaged—the ratchet member 8 can only be rotated in one direction with respect to the housing 4. Preferably, the movement of the ratchet member 8 corresponding to that of the ratchet feature 21 passing one tooth of the teeth 22 relates to the setting of a minimum settable dose of drug 35.

The drive assembly is configured such that in the setting mode of operation, the releasable uni-directional coupling is established and, in the dispensing mode of operation, the releasable uni-directional coupling is disengaged and the ratchet member 8 rotates in the first direction with respect to the housing 4 such that also the drive member 5 is rotated in the first direction with respect to the housing 4. The arrow 39 indicates the second direction according to which the dose setting member 11 is rotated with respect to the housing 4 during a setting action, whereby a dose of drug 35 of the drug delivery device 100 is set.

FIG. 3 shows a cross-section of the proximal end 18 of the housing 4. One can observe the teeth 22 at the inside of the housing 4. The teeth 22 interact with the ratchet feature 21, in order to form the releasable unidirectional coupling, as mentioned above.

When, in the setting mode of operation, the actuation button 12 is depressed with respect to the housing, the ratchet features 21 are pulled radially inwards by a finger (not shown) on the clutch element 10, allowing the ratchet member 8 to rotate due to a driving force of the biased resilient member 13. The ratchet features 21 may take the form of a loop and the finger on the clutch element 10 may interact with the inner surface of said loop, thus pulling the ratchet features 21 radially inwards and disengaging them from the teeth 22.

The housing 4 further comprises a setting stop 23 and a dispensing stop 24. These components prevent under certain conditions a rotation of the ratchet member 8 with respect to the housing 4 in the proximal and in the distal direction, respectively. Said stops may engage with corresponding stops on the ratchet member 8, respectively. A corresponding setting stop may, e.g. be arranged at a proximal end of the outer thread 31 of the ratchet member 8 and a corresponding dispensing stop may be arranged at a distal end of the outer thread 31. Thus, between certain limits, the ratchet member 8 is rotationally moveable with respect to the housing 4.

FIG. 4 shows inner components of the drug delivery device 100. Particularly, a last dose feature 25 of the dose member 9 is shown which is arranged to engage a piston rod feature 43 of the piston rod 7. During a setting action, the dose member 9 preferably rotates in a second direction with respect to the housing 4 and with respect to the drive member 5. When, in the setting mode of operation—whereby the dose member 9 moves axially away from the distal end 19—the maximum settable dose of drug 35 is reached. In the setting mode of operation, the axial distance by which the dose member 9 may be moved proximally is equal or less than the axial distance between the last dose feature 25 and the piston rod feature 43. The axial distance between the piston rod feature 43 and the last dose feature 25 is indicative for an amount of drug 35 remaining to be dispensed from the drug delivery device 100. When the last dose feature 43 and the piston rod feature 25 are engaged, the dose member 9 is prevented from being moved further proximally. The last dose feature 25 prevents the user from setting a dose greater than the one corresponding to the available volume in the cartridge 1.

The drive assembly comprises a drive coupling being configured such that in the setting mode of operation, the ratchet member 8 is free to rotate with respect to the dose member 9. In this situation, the drive coupling is preferably disengaged. The drive coupling is further configured such that the dispensing mode of operation, the ratchet member 8 is rotationally locked with respect to the dose member 9 such that, during a dispensing action, the dose member is rotated in the first direction with respect to the housing 4. In this situation, the drive coupling is, preferably engaged.

In order to achieve the drive coupling, the ratchet member 8 comprises ratchet splines 27 which engage corresponding dose member splines 33 of the dose member 9 when the actuation member 12 is depressed.

FIG. 5 shows selected components of the drug delivery device 100 by means of a sectional perspective view. In the depicted situation, the actuation member 12 is not pressed such that the setting mode of operation is indicated. Particularly, the ratchet splines 27 of the ratchet member 8 are shown which are configured to engage the dose member splines 33 when it is switched to the dispensing mode of operation. The actuation member 12 comprises an actuation pin 49 by which the actuation member 12 and the dose setting member 11 are rotationally locked. When the actuation member 12 is pressed, the actuation pin 49 pushes the clutch element 10 in the distal direction, thereby distally displacing the clutch element 10 such that ratchet splines 27 disengage the dose member splines 33 of the dose member 9 and the releasable uni-directional coupling is released.

FIG. 6 shows the selection member 3 which forms part of and display assembly of the drug delivery device 100 (cf. FIG. 8). The selection member 3 is a selection sleeve comprising a non-continuous helical cut-out.

FIG. 7 shows in the indication member 2 further comprising indicia 28. The indication member 2, as well forms a part of the mentioned display assembly of FIG. 8. The indicia 28 may comprise dose numbers indicating quantities or units of drug 35 to be dispensed from the drug delivery device 100. The indicia 28 may be arranged helically around the circumference of the indication member 2. The indicia may extend for example from “zero” at a distal end of the indication member 2 to the maximum allowable dose, for example “120” at a proximal end of indication member 2. The drug delivery device 100 further comprises a coupling member 29. The indication member 2 further comprises an indication member thread 48 to which a coupling member 29 (cf. FIG. 8) may be threadedly engaged. The coupling member 29 may be a sliding window sliding axially along the window 17. The indication member 2 further comprises an axial rib 38 by which the indication member 2 may be rotationally locked with respect to the ratchet member 8. Therefore, the ratchet member 8 may comprise a corresponding notch (not explicitly indicated) engaging the axial rib 38.

FIG. 8 shows at least parts of the display assembly of the drug delivery device 100 comprising the indication member 2, the selection member 3 and the coupling member 29. The coupling member 29 is threadedly engaged to the indication member thread 48. The coupling member 29 is further rotationally locked but axially moveable with respect to the housing 4. When, during an operation of the drug delivery device 100, the indication member 2 is rotated by way of a rotation of the ratchet member 8, the coupling member 29 is moved axially with respect to the housing, thereby driving or rotating the selection member 3 with respect to the indication member 2. Said rotation of the selection member 3 occurs in the same direction in which also the indication member 2 has been rotated previously.

At rest or in an initial position, the display assembly preferably displays “0” or an equivalent marking to show that no dose has been set.

An application of the display assembly is not bound to drug delivery devices but may relate to any conceivable delivery device or further applications.

The selection member 3 defines a masking section 45 and a non-masking section 46. A non-masking section 46 and the masking section 45 partially overlap with the window 17 which is indicated dashed in FIG. 8. A movement of the indication member 2 with respect to the housing 4 is converted into rotational movement of the selection member 3 with respect to the indication member 2.

The display assembly may be configured such that the indication member 2 and the selection member 3 are coupled such that they rotate in the same direction or in opposite directions, particularly when one of these members is moved with respect to the other one of these members.

The coupling member 29 comprises a coupling member window 47. Due to the interaction of the indication member, the selection member and the coupling member, only a limited amount of information is displayed to the user, thus preventing confusion during an operation of the drug delivery device 100. The masking section 45 may be formed by a body of the selection member 3.

The selection member 3 and the indication member 2 are preferably coupled such that movement of the indication member with respect to the housing 4 is converted into rotational movement of the selection member 3 with respect to the indication member 2.

Alternatively, the selection member 3 and the indication member 2 are preferably coupled such that rotational movement of the selection member 3 with respect to the housing 4 is converted into movement of the indication member 2 with respect to the selection member 3.

The non-masking section 46 and the window 17, particularly in conjunction with the masking section 45, may define a displayed section 52 of the indication member 2. For this purpose, the non-masking section 46 and the masking section 45 may partially, preferably only partially, overlap with the window 17 to define the displayed section 52 of the indication member 2. As the masking section 45 also partially overlaps with the window 17 as well as the non-masking section 46, it is guaranteed that only a selected portion of the indication member 2 is displayed through the window 17. The remaining portions of the indication member 2 which would be visible in the window 17, as the window 17 may have a significant axial extension, are masked by the masking section 45 of the selection member 3.

The displayed section 52 of the indication member 2 may be axially displaced within the window 17.

Preferably, the non-masking section 46 is arranged between two portions of the masking section 45, particular as seen along the window axis. One of the portions of the masking section 45 is expediently arranged more distally than the other one. The portions may delimit the non-masking section 46. These portions of the masking section 45 may be provided with different markings, for example with different colours.

In the situation depicted in FIG. 8, the number “10” is displayed through the coupling member window 47 and the window 17. The window 17 may be covered either by a window insert connected to the body or by a label, preferably a transparent label. Said body insert may be a magnifying insert and shaped like a lens in order to magnify or increase the apparent size of the indicia 28 on the indication member 2.

As an alternative to the coupling member, the selection member 3 could also be driven relative to the indication member 2 by, e.g., a spur gear train.

As mentioned above, the display assembly is configured such that the indication member 2 and the selection member 3 preferably do not move axially with respect to the housing. This provides the advantage of a compact embodiment of the drug delivery device 100, as compared to devices which require e.g. large axial displacements of the respective indication sleeves. In the presented concept, the geometry of the indicia is not directly tied to the geometry of the drive assembly. Thereby, the indicia may be embodied comparably large.

Another advantage of the described display assembly relates to the fact that the position of the coupling member window 47 moves axially when the device is operated. This gives particularly a visual cue to the user that the set dose is increased during a setting action and the dose to be dispensed is decreased during a dispensing action.

The indicia 28 do not necessarily comprise numbers. The indicia could also comprise visual indicators, e.g. colours, digits, numbers, letters, words, written text, graphics, icons and/or combinations thereof. In addition to the information provided by the indicia 28, an external face of the indication member 2 could be coloured, e.g. red at in a distal region and green in a proximal region. The indication member 2 may be configured such that when the set dose is increased, the length of the red region which is visible through the window 17 is increased and the length of the green region is reduced. Said colours may also be varied or changed.

FIG. 9 shows inner components of the drug delivery device 100 in the dispensing mode of operation, wherein the actuation member 12 is depressed, i.e. moved distally with respect to the housing 4. When, originating from the setting mode of operation, the actuation member 12 is moved distally with respect to the housing 4, the actuation member 12 rotationally locks in the auxiliary drive member 26 via actuation member splines 32. The clutch element 10 may comprise proximal teeth 51 which—in the setting mode of operation—engage distal teeth 53 of the dose setting member 11 by way of the clutch spring 14. Thus, setting mode of operation, the dose setting member 11 and the clutch element 10 are rotationally locked by the proximal teeth 51 and the distal teeth 53.

For the rotational locking between the dose setting member 11 and the ratchet member 8, the dose setting member 11 has distal teeth 53 which engage with proximal teeth 51 provided by the clutch element 10. The clutch element 10 may have a dose setting finger (not explicitly indicated) which is configured to engage the ratchet member 8.

In the setting mode of operation, the ratchet member 8 is free to rotate with respect to the dose member 9. The ratchet member 8 is further rotationally locked with respect to the dose setting member 11. During setting of a dose, the clutch element 10 rotates along with the ratchet member 8 and the dose setting member 11. During delivery of a dose, the clutch element 10 rotates with the ratchet member 8 without rotating with the dose setting member 11 which is rotationally fixed with respect to the housing 4. When a user rotates the dose setting member 11 in order to set a dose, the ratchet member 8 rotates in the second direction with respect to the housing 4 and simultaneously moves proximally with respect to the housing 4 due to the threaded engagement thereto (cf. threads 30 and 31). Thereby, the resilient member 13 is biased and a relaxation of the resilient member or a back rotation is prevented by the releasable uni-directional coupling between the ratchet member 8 and the housing 4.

When the user then presses the actuation member 12 with respect to the housing 4, it may have a small travel of, e.g. 1 mm in which no action takes place. When the actuation member 12 has been moved by the full distance (cf. FIG. 1) in the distal direction with respect to the housing 4, it is switched from the setting mode to the dispensing mode of operation, as mentioned above. Moreover, the releasable uni-directional coupling is released and the ratchet member 8 is now free to rotate with respect to the housing 4. Simultaneously, the ratchet member 8 is rotationally locked to the dose member 9 via the mentioned drive coupling. Moreover the dose setting member 11 is rotationally locked with respect to the housing 4 via the actuation features 20 of the actuation member 12. Consequently, the spring energy of the resilient member 13 which is stored during setting of the dose is exerted to the ratchet member 8 which, thus, rotates in the first direction with respect to the housing 4. As said rotation of the ratchet member 8 being rotationally locked with respect to the dose member 9 is further transferred to drive member 5 and the piston rod 7, said piston rod 7 rotates in the first direction. Due to the threaded engagement of the piston rod 7 with the nut member 15, the plunger 16 is thus advanced within the cartridge 1 in order to dispense the set dose of drug 35 from the drug delivery device 100.

Additionally, the pressure exerted by the user to the actuation member 12 may be directly exerted to the drive member 5 via the auxiliary drive member 26. When the user depresses the actuation member 12, the auxiliary drive member 26 is rotationally locked with respect to the actuation member 12 by actuation member splines 32 and pressure is transferred or converted into a rotation of the drive member 5 with respect to the housing 4 due to the threaded engagement of the auxiliary drive member 26 and the drive member 5. Therefore, the stronger the user presses the actuation member 12, the more torque is exerted to the drive member 5 and the faster the piston rod 7 will rotate, thereby assisting the advancement of the plunger 16 in the cartridge 1 during the dispensing action. This functionality characterizes the auxiliary drive mechanism. When or before the actuation member 12 abuts a proximal face of the dose setting member 9 during the axial travel of the actuation member 12, the auxiliary drive member 26 may abut an actuation stop (not explicitly indicated) such that an axial force may be exerted to the auxiliary drive member 26.

The problems with spring-driven drug delivery devices are that the user has no control over the speed of dispensing or injection and the user has little haptic feedback of the injecting or dispensing progress because the thumb by which the actuation member 12 is preferably actuated according to the presented concept, does not move. The presented concept may be suitable for the dispensing of 120 units of insulin formulation or a different drug, whereby the user is allowed to control the dispensing and whereby haptic feedback is provided from the movement of the actuation member 12.

The end of a dispensing or injection action may be indicated to the user by a feature which e.g. provides for an acoustic feedback when two components move relative to one another at the end of the dispensing action. If an acoustic feedback is required, every time the display assembly displays zero doses, then, the mentioned feature could be provided by the indication member, the selection member, the coupling member or the ratchet member, for example. If the acoustic feedback is required only once at the end of a dispensing action (and not during a setting action), said feature could be provided at the drive member or the dose member such that a relative movement between these components provides the acoustic feedback.

When, once the actuation member 12 has been pressed, the axial or distal force on the actuation member 12 is removed, the actuation member 12 returns to its initial axial position relative to the dose setting member 11. Thereby, the ratchet member 8 is again engaged with the housing 4 via the releasable uni-directional coupling, thus preventing further dispensing. The actuation features 20 are furthermore pulled radially inwards such that the dose setting member 11 is rotatable with respect to the housing 4 again.

FIG. 10 shows inner components of the drug delivery device 100 by means of a respective sectional view. Particularly, FIG. 10 shows the piston rod 7 comprising a thread 36 in greater detail. The piston rod 7 and also further components of the drive assembly or the drug delivery device 100 are shown sectional, only. One can see that the thread 36 does not extend along the whole circumference of the piston rod 7. Instead, the thread 36 is only formed or embodied on opposing sides of piston rod 7 (only one side is shown due to the sectional view). According to this embodiment, the piston rod 7 may be formed from sheet metal. As compared to e.g. piston rods made from moulded thermoplastics, the use of sheet metal allows smaller tolerances. Thermoplastics, on the other hand, are liable to creep if a load is provided on them for extended durations. Furthermore, the magnitude of force transferred through plastic plunger rods may be limited by the material strength. Indeed, the high density of steel can lead to breakages during drop testing. The reduced mass of a plunger rod manufactured from sheet metal compared with a conventional plunger rod, however, should reduce the number and severity of failures e.g. in drop testing.

Moreover, the reduced mass accompanied by the use of sheet metal allows material cost savings. Additionally, the processes involved in the manufacture of sheet metal are typically less time-consuming and, therefore, cheaper. Additionally, less waste of material is created, as compared to conventional machining processes.

Two or more plunger rods manufactured from sheet metal could be stacked to create, e.g. a “conventional” bulk manufactured plunger rod. For example, two or more plunger rods which are fabricated from sheet metal may be axially sprung relative to one another such that they provide an anti-backlash functionality.

The drive member 5 further comprises a drive member support 34, thus supporting the piston rod 7 at lateral sides of the piston rod 7 not comprising the thread 36 within a bearing of the nut member 15. Due to the support 34, the drive member 5 cannot move away from the main axis of the drug delivery device 100 and radial movement of the piston rod 7 is prevented.

The drug delivery device 100 may need to be primed, i.e. the act of preparing the device for the first use. Priming may relate to setting and delivering one or more small doses into air so that any play, clearances or tolerances in the drug delivery device 100 are removed and the components are brought into suitable compression or tension. After first use, and before each subsequent use, a ‘safety shot’ may be dispensed into air to ensure that the needle is not blocked.

The drug delivery device 100 may be used to dispense or inject a drug, a substance such as a liquid medicament. The steps the user may have to carry out in this respect comprise the removal of the cap, the fitting of the needle, the dialling or setting of a priming dose—which may comprise two units of insulin formulation—by rotating the dose setting member 11, the dispensing of the set priming dose by pressing the actuation button 12, the setting of a dose which is actually to be dispensed for the drug delivery device 100 by rotating the dose setting member 11, inserting the needle, dispensing the set dose by pressing the actuation member 12 and the removal of the needle from the device and replacing the cap to the drug delivery device 100.

Actually, layout, function and number of components of the drive assembly may differ from the illustrations presented in the figures.

The drug delivery device 100 may be disposable such that the cartridge 1 cannot be replaced. Alternatively, the drug delivery device 100 may be reusable, wherein the cartridge 1 may be replaced or removed from a cartridge holder of the drug delivery device 100 or the drug delivery device 100. Furthermore, the drive assembly may be configured such that the piston rod 7 is resettable which may be expedient if the drug delivery device 100 is reusable.

The display assembly is preferably configured such that when a minimum settable dose is set, the spring energy of the resilient member 13 stored during the setting action is sufficient to deliver said minimal dose.

Although not explicitly indicated and described, the drive assembly may be configured such that a set dose may be changed or decreased, e.g. by rotating the dose setting member 11 in the first direction once a dose has been set. To this effect, the teeth 22 and the ratchet features 21 may be configured accordingly. In this case, the releasable uni-directional coupling is to be replaced by, e.g. a releasable bi-directional coupling.

A “hold time” is the period from when the drive mechanism and/or the auxiliary drive mechanism has stopped moving—this may be the case when the display assembly displays “0”—to when the dose is fully dispensed. The hold time is required, as, if a user dispenses the drug too fast, it takes some time until the elasticity of components of the drive assembly equilibrate such that the correct volume of drug 35 is delivered.

Motor-driven drug delivery devices often cause problems, as the motors are expensive, the motors are heavy, the motors require a power supply such as batteries which add further cost, and as the motors comprise considerable weight and cause environmental impacts on disposal. Furthermore, motors normally require electronic control systems which increase the cost, complexity, regulatory challenge.

The described display assembly can be used in any device which would benefit from displaying the relative position of components, especially if those components rotate relative to one another. The most relevant applications are in dispensing mechanisms, such as drug delivery devices like pen-type devices or injection-type devices, medical devices such as dispensers of antiseptic creams, analgesic creams, detergents and so on. Furthermore, this holds for applications in devices for dispensing adhesives, lubricants, paints, detergents and such like. Another example may relate to food dispensers for non-rigid foods such as e.g. tomato sauce, crushed garlic, cheese, butter, juice, smoothies, soup, coffee, tea, jam, peanut butter.

The described leadscrew may relate to any application which currently uses a plunger rod.

The term “drug”, “substance” and/or “liquid medicament”, as used herein, preferably means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin;

human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivatives are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropin (Follitropin, Lutropin, Choriongonadotropin, Menotropin), Somatropin (Somatropin), Desmopressin, Terlipressin, Gonadorelin, Triptorelin, Leuprorelin, Buserelin, Nafarelin, Goserelin.

A polysaccharide is for example a glucosaminoglycan, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The scope of protection of the invention is not limited to the examples given hereinabove. The invention is embodied in each novel characteristic and each combination of characteristics, which particularly includes every combination of any features which are stated in the claims, even if this feature or this combination of features is not explicitly stated in the claims or in the examples.

### REFERENCE NUMERALS

1 Cartridge

2 Indication member

3 Selection member

4 Housing

5 Drive member

7 Piston rod

8 Ratchet member

9 Dose member

10 Clutch element

11 Dose setting member

12 Actuation member

13 Resilient member

14 Clutch spring

15 Nut member

16 Plunger

17 Window

18 Proximal end

19 Distal end

20 Actuation feature

21 Ratchet feature

22 Teeth

23 Setting stop

24 Dispensing stop

25 Last dose feature

26 Auxiliary drive member

27 Ratchet spline

28 Indicium

29 Coupling member

30 Inner thread (housing)

31 Outer thread (ratchet member)

32 Actuation member spline

33 Dose member spline

34 Drive member support

35 Drug

36 Thread (piston rod)

38 Axial rib

39 Second direction

43 Piston rod feature

45 Masking section

46 Non-masking section

47 Coupling member window

48 Indication member thread

49 Actuation pin

50 Distal termination

51 Proximal teeth

52 Displayed section

53 Distal teeth

100 Drug delivery device

x Longitudinal axis

1. Drive assembly for a drug delivery device (100), comprising:
a housing (4) having a proximal end (18) and a distal end (19),
a main drive mechanism comprising a drive member (5), a piston rod (7) being coupled to the drive member (5) and a resilient member (13), the main drive mechanism being configured such that, in a setting mode of operation, the resilient member (13) is biased when a dose of a drug (35) is set and, in a dispensing mode of operation, the set dose is dispensed from the drug delivery device (100) in a dispensing action, wherein the drive member (5) drives the piston rod (7) with respect to the housing (4), the dispensing action being driven by the resilient member (13) and
an auxiliary drive mechanism comprising an auxiliary drive member (26) which is coupled to the drive member (5), wherein the auxiliary drive mechanism is configured such that, in the dispensing mode of operation, the auxiliary drive mechanism can be actuated by a user via a movement of the auxiliary drive member (26) with respect to the housing (4), thereby mechanically assisting the dispensing action of the main drive mechanism.

2. Drive assembly according to claim 1, wherein the drive member (5) is coupled to the resilient member (13) and in the dispensing mode of operation, the drive member (5) is rotatable in a first direction with respect to the housing (4).

3. Drive assembly according to claim 1 or 2, comprising an actuation member (12) which is coupled to the auxiliary drive member (26) and axially movable with respect to the housing (4), wherein the drive assembly is configured such that when, in the setting mode of operation the dose is set and the actuation member (12) is moved distally with respect to the housing (4), the drive assembly is switched from the setting mode to the dispensing mode of operation and the auxiliary drive member (26) is moved distally with respect to the housing (4), thereby transferring a force to the drive member (5) in order to drive the drive member (5) in addition to the force provided by the resilient member (13).

4. Drive assembly according to claim 2 or 3, wherein the drive assembly is configured such that when, in the setting mode of operation, a dose is set, the auxiliary drive member (26) is rotated with respect to the drive member (5) in a second direction, opposite to the first direction and when in the setting mode of operation the dose is set and the actuation member (12) is moved distally with respect to the housing (4), the drive member (5) rotates in the first direction with respect to the auxiliary drive member (26).

5. Drive assembly according to claim 3 or 4, wherein the drive member (5) is threadedly engaged to the auxiliary drive member (26) and rotationally locked with respect to the piston rod (7) and the piston rod (7) is threadedly engaged to a housing component (15) of the drive assembly, wherein the drive assembly is configured such that when, in the setting mode of operation the dose is set and the actuation member (12) is depressed by the user in order to be moved distally with respect to the housing (4), the auxiliary drive member (26) mechanically assists a rotation of the drive member (5), whereby the piston rod (7) is moved distally with respect to the housing (7).

6. Drive assembly according to at least one of the previous claims, comprising a dose member (9), wherein the dose member (9) is rotationally locked with respect to the drive member (5) and wherein the drive assembly is configured such that, the dose member (9) is moved proximally with respect to the drive member (5) when a dose is set.

7. Drive assembly according to claim 6, wherein the dose member (9) comprises a last dose feature (25) which is arranged to engage a piston rod feature (43) of the piston rod (7) when, in the setting mode of operation, the axial distance by which the dose member (9) is moved proximally is less than the axial distance between the last dose feature (25) and the piston rod feature (43), the axial distance between the piston rod feature (43) and the last dose feature (25) being indicative for an amount of drug (35) remaining to be dispensed from the drug delivery device (100), wherein, when the last dose feature (25) and the piston rod feature (43) are engaged, the dose member (9) is prevented from being moved further proximally.

8. Drive assembly according to at least one of the claim 4, 5 or 7, comprising a ratchet member (8) being coupled to the housing (4) via a releasable uni-directional coupling, wherein the drive assembly is configured such that, in the setting mode of operation, the releasable uni-directional coupling is established and the ratchet member (8) is rotated in the second direction with respect to the housing (4) when a dose is set, whereby the ratchet member (8) is moved proximally with respect to the housing (4) and with respect to the drive member (5) and wherein in the dispensing mode of operation, the releasable uni-directional coupling is disengaged and the ratchet member (8) rotates in the first direction with respect to the housing (4).

9. Drive assembly according to claim 8, comprising a drive coupling which is configured such that in the setting mode of operation, the ratchet member (8) is free to rotate with respect to the dose member (9) and, in the dispensing mode of operation, the ratchet member (8) is rotationally locked with respect to the dose member (9) such that, during a dispensing action, the dose member (9) is rotated in the first direction with respect to the housing (4).

10. Drive assembly according to claim 9, comprising a clutch element (10) and a clutch spring (14), wherein the clutch spring (14) is retained between the ratchet member (8) and the clutch element (10), thereby tending to move the ratchet member (8) and the clutch element (10) away from each other, wherein the drive assembly is configured such that when, in the setting mode of operation, the actuation member (12) is moved distally with respect to the housing (4), the clutch element (10) is moved distally, such that the drive coupling is engaged.

11. Drive assembly according to at least one of the claims 8 to 10, wherein the resilient member (13) is a torsion spring, wherein the resilient member (13) is connected to the ratchet member (8) such that it is biased when the ratchet member (8) is rotated in the second direction with respect to the housing (4).

12. Drive assembly according to at least one of the claims 8 to 11, comprising a dose setting member (11), wherein the actuation member (12) is axially movable with respect to the dose setting member (11), wherein the drive assembly is configured such that, in the setting mode of operation, the dose setting member (11) is rotationally locked with respect to the ratchet member (8) and when, in the setting mode of operation, the actuation member (12) is moved distally with respect to the housing (4) and the dose setting member (11), the rotational locking between the ratchet member (8) and the dose setting member (11) is released and the dose setting member (11) is rotationally locked with respect to the housing (4) by an actuation member feature (20) of the actuation member (12).

13. Drive assembly according to claim 12, wherein the drive assembly is configured such that, the dose setting member (11) is rotated by the user in the second direction with respect to the housing (4) in order to set a dose.

14. Drive assembly according to at least one of the claims 8 to 13, wherein, the drive assembly is configured such that, in the setting mode of operation, the releasable uni-directional coupling is releasable when the dose setting member (11) is rotated in the second direction with respect to the housing (4).

15. Drive assembly according to at least one of the previous claims, wherein the piston rod (7) comprises a metal and a rectangular cross section.

16. Drive assembly according to at least one of the previous claims, wherein distal movement of the auxiliary drive member (26) can be transferred into a rotation of the drive member (5).

17. Drive assembly according to at least one of the previous claims, wherein the drive member (5) is threadedly engaged to the auxiliary drive member (26).

18. Drive assembly according to at least one of the previous claims, wherein the drive member (5) is rotationally locked with respect to the piston rod (7).
